(19) United States
(12) Patent Application Publication
(10) Pub. No.: US 2023/0169473 A1
(43) Pub. Date: Jun. 1, 2023

(54) MEDICATION RETURN PLATFORM FOR EXTRACTING VALUE FROM UNUSED MEDICATIONS

(71) Applicant: THE BOARD OF REGENTS OF THE UNIVERSITY OF TEXAS SYSTEM, Austin, TX (US)

(72) Inventor: Darin T. OKUDA, Dallas, TX (US)

(73) Assignee: THE BOARD OF REGENTS OF THE UNIVERSITY OF TEXAS SYSTEM, Austin, TX (US)

(21) Appl. No.: 17/919,639

(22) PCT Filed: Apr. 22, 2021
(86) PCT No.: PCT/US2021/028694

§ 371 Date: Oct. 18, 2022

Related U.S. Application Data
(60) Provisional application No. 63/013,880, filed on Apr. 22, 2020.

(51) Int. Cl. G06Q 10/30; G06Q 10/0837; G06Q 30/0234; G16H 20/10; G16H 50/20; G06V 20/64; G06V 30/18; H04L 9/00

(52) U.S. Cl. CPC G06Q 10/30; G06Q 10/0837; G06Q 30/0234; G16H 20/10; G16H 50/20; G06V 20/64; G06V 30/18; H04L 9/50; G06Q 2220/00

(57) ABSTRACT

Described herein is a medication return platform for managing returns of unused medications. In various embodiments, the medication return platform authorizes shipments of unused medications to manufacturers for disposal. The medication return platform may also provide a rebate for the returned medication to health insurance companies, employers, and other purchasers of medication. To facilitate the return process, the medication return platform may provide de minimis gifts to patients in exchange for their unused medications.

### RELATED APPLICATIONS/PRIORITY CLAIMS

This application claims the benefit under 35 USC 119(e) and 120 of U.S. Provisional Application No. 63/013,880 filed Apr. 22, 2020, and which is incorporated herein by reference. This application is related to PCT Application No. PCT/US20/18016 filed Feb. 13, 2020, which claims priority to U.S. Provisional Application No. 62/804,848 filed Feb. 13, 2019, and both applications are incorporated herein by reference.

### FIELD

The disclosure relates generally to systems and methods of managing the return and disposal of unused pharmaceuticals. This disclosure also relates generally to systems and methods of monitoring the use of pharmaceutical therapies.

### BACKGROUND

Treating life altering medical conditions is extremely expensive. Patients, employers, insurance companies, and other payers must cover costs for treatment including disease modifying therapies (DMTs), prescribed symptomatic treatments, scheduled and unscheduled office visits, urgent care or emergency room evaluations, hospitalizations, physical, occupational, or speech therapies, supportive devices, healthcare costs associated with adverse reactions to prescribed immunomodulatory or immunosuppressive treatments, long-term care, and the like. In many cases, the most significant costs are obtaining new, state of the art pharmaceuticals and other DMTs. Manufacturers justify the high costs of DMTs based on the tremendous R&D costs associated with new drug development, the low success rate of drug development programs, and the ability of a new agent to dramatically transform the treatment landscape and offset other healthcare costs.

Manufacturers may have near monopoly power to charge whatever they want for newly developed drugs for a limited period of time. Therefore, the cost of DMTs are well above inflation. For example, the increase in annual costs for multiple sclerosis (MS) DMTs is about 5-7 times higher than prescription drug inflation. Between 2008 and 2012, the sales of the available treatments for MS increased from $4 billion to approximately $9 billion annually. For many conditions, several treatments exist and most patients must try more than one drug before discovering an effective treatment for their condition. The tendency of patients to switch between existing therapies and try new drugs once they are approved compounds the high cost of DMTs.

Once a physician prescribes a particular DMT, patients are typically able to acquire a 1 to 3-month supply of the prescribed drug. In the event a decision is made to switch to another treatment, the remaining supply of the previously filled prescription is wasted. Typically, the amount of unused medication that a patient may have may range from 1 to 89 days. In many cases, patients may receive a refill for their treatment a week into their prescription. Therefore, in many instances, patients may obtain more than 90-day surplus supply of medication. Unused medication may also be obtained by patients who continue on a treatment while waiting for insurance approval for their next DMT. These patients may need to refill their original drug even after they are prescribed a new treatment because timely approvals of new DMTs by insurance companies are uncertain. Once the new DMT is approved, the patient may be stuck with an excess supply of the original drug. Additionally, some patients may continue to refill their medication without taking it. Patients who represent to family and their physician that they are taking their medication without actually taking it may have up to six months or more of unused medication.

Once distributed to patients, the use and or disposal of unused medications is unregulated and there are no existing platforms for providing reimbursement or other incentives for collecting controlled substances and other unused pharmaceuticals. Therefore, unused DMTs and other medications are commonly discarded in the trash, flushed down the toilet, stored within refrigerators in the homes of patients, and the like. Additionally, within some condition support groups, DMTs may be given to other members to use or even to try before a treatment switch is requested by their healthcare provider. In other instances, unused drugs are exchanged illegally on online forums or sold illegally on the street.

The proper disposal and removal of unused medication from circulation is a critical component of the drug lifecycle. The opioid epidemic is a recent example of a public health crisis that can emerge if unused medications are not safely destroyed. Additionally, discarding drugs in the trash and/or flushing medications by patients who have no experience with chemical waste and/or knowledge of drug specific disposal guidelines leads to harmful environmental impacts. DMTs and other medications include powerful chemicals that can significantly alter biological systems. Drug metabolites can have significant impacts on animals and microorganisms within the environment. Despite highly effective water treatment facilities, small concentrations of pharmaceuticals have consistently been found in our water sources and treated drinking water. Few studies have investigated the long term impact of exposure to trace amounts of DMTs so unique combinations of various compounds could have unanticipated effects on humans, wildlife and other aspects of the environment. Despite the importance of proper disposal of unused medications, no platforms for collecting and destroying unused medications currently exist.

Additionally, the true prevalence of patients switching to another disease modifying therapy is unknown but the current evidence suggests that approximately 35% of patients transition to another treatment annually. Testing DMTs that are not approved by healthcare providers can lead to dangerous side effects. With no feasible, efficient mechanism for predicting drug discontinuation or returning and disposing unused DMT tens of millions of dollars worth, if not more, of unused DMTs are wasted every year. Accordingly, there is a well-established need for a medication return platform that facilitates collecting and properly destroying unused medications. There is also a well-established need for a persistent monitoring application at can track patient use data for medications and predict drug discontinuations based on the use data.

### BRIEF DESCRIPTION OF THE FIGURES

FIG. 1 illustrates an exemplary process for managing the return of unused medications, according to various embodiments of the present disclosure.

FIG. 2 illustrates an exemplary system for managing returns of unused medications, according to various embodiments of the present disclosure.

FIG. 3 illustrates more details of the system shown in FIG. 2, according to various embodiments of the present disclosure.

FIGS. 4A-G illustrate exemplary graphical user interfaces (GUIs) for collecting medication return data according to various embodiments of the present disclosure.

FIGS. 5A-D illustrate exemplary GUIs for generating a shipping label and shipping unused medications according to various embodiments of the present disclosure.

FIG. 6 illustrates more details of the persistent monitoring application shown in FIG. 2, according to various embodiments of the present disclosure.

FIGS. 7A-E illustrate exemplary use monitoring GUIs, according to various embodiments of the present disclosure.

FIG. 8 illustrates exemplary survival plots for medications, according to various embodiments of the present disclosure.

FIG. 9 is a flow chart illustrating an exemplary method of returning unused medications, according to various embodiments of the present disclosure.

FIG. 10 is an exemplary computer system implementing a client device or other computer implemented component of the exemplary system shown in FIG. 2 according to various embodiments of the present disclosure.

### DETAILED DESCRIPTION OF SEVERAL EMBODIMENTS

Disclosed herein are systems and methods for recovering, returning, and managing, partially used and unused medications for a variety of conditions. In various embodiments, the medication return platform facilitates collaboration between patients, healthcare providers, pharmacies, insurance providers, government agencies, government healthcare systems, drug manufacturers and other pharmaceutical industry stakeholders to manage the collection and disposal of used medications. The return platform may automate aspects of the drug collection, verification, disposal, and reimbursement process to enable unused medications to be efficiently returned to manufacturers. The return platform may also include a notification system for notifying drug manufacturers, healthcare providers, insurance companies, and pharmacies when a patient switches to a new medication to reduce the amount of unwanted medications distributed to patients. Data collected by the return platform may also be used to train one or more machine learning models. The predictions generated by the machine learning models and the data collected by the return platform may be shared with subscribers of the medication return platform. Subscribers may include, for example, patients, healthcare providers, health insurance companies, pharmacies, pharmaceutical manufacturers, pharmacy benefit managers, government agencies, and the like. The predictions and data distributed to the subscribers may be used to develop more effective treatments for patients, track the effectiveness and side effects of therapies, determine the number of patients switching from and/or using a particular medication, streamline insurance approval for new medications, improve the efficiency of drug manufacturing supply chains, more precisely manage pharmaceutical inventories, and the like.

The return platform may include one or more legal incentives to encourage the return of access medications. For example, the platform may provide a rebate in exchange for any collected medications. Rebates may be provided to patients that return medications in the form of a discount for the purchase of future medications. Rebates and or other incentives provided to patients returning excess medications may be distributed using a value-based contract. Value based contracts may be smart contracts deployed on one or more blockchains and or distributed ledgers. The smart contracts may execute autonomously to distribute rebates or other incentives based on receiving one or more pieces of data by the return platform. For example, a rebate smart contract may distribute a rebate automatically to a patient's account upon receiving, for example, at least one of an image of the returned medication, the patient's account information, the patient's insurance information, a medication currently prescribed to the patient, and a certification that the returned medication was collected and or meets a quality collection standard.

As described herein, the terms “medication”, “medications”, “drug”, and “drugs” may refer to any DMT and/or symptomatic or preventative treatment for any condition including, for example, multiple sclerosis, glioblastoma multiforme, Parkinson's disease, rheumatoid arthritis, systemic lupus erythematosus, psoriatic arthritis, ankylosing spondylitis, multiple myeloma and other forms of cancer, neuromyelitis optica spectrum disorder, sleep disorders, cystic fibrosis, hematological malignancies, Alzheimer's disease and other neurodegenerative or neurological conditions, hemophilia, hepatitis, HIV/AIDS, Crohn's disease, ulcerative colitis, solid organ tumors, organ transplants, hereditary amyloidosis, metabolic deficiencies, hormonal deficiencies, pulmonary hypertension, hypercholesterolemia, spinal muscular atrophy, muscular dystrophies, and other muscle disorders, and any condition resulting in pain managed using an opioid or other pain killing medication.

As described herein, the term “medical information” may refer to treatment condition history and treatment history relating to any condition including, for example, multiple sclerosis, glioblastoma multiforme, Parkinson's disease, rheumatoid arthritis, systemic lupus erythematosus, psoriatic arthritis, ankylosing spondylitis, multiple myeloma and other forms of cancer, neuromyelitis optica spectrum disorder, sleep disorders, cystic fibrosis, hematological malignancies, Alzheimer's disease and other neurodegenerative or neurological conditions, hemophilia, hepatitis, HIV/AIDS, Crohn's disease, ulcerative colitis, solid organ tumors, organ transplants, hereditary amyloidosis, metabolic deficiencies, hormonal deficiencies, pulmonary hypertension, hypercholesterolemia, spinal muscular atrophy, muscular dystrophies, and other muscle disorders, and any condition resulting in pain managed using an opioid or other pain killing medication.

As described herein, the term “medication information” may refer to any information describing or related to medication returned by a patient including for example, a medication description, patient information, patient insurance information, medication information, treatment start date, and other medical information related to the returned medication, and the like.

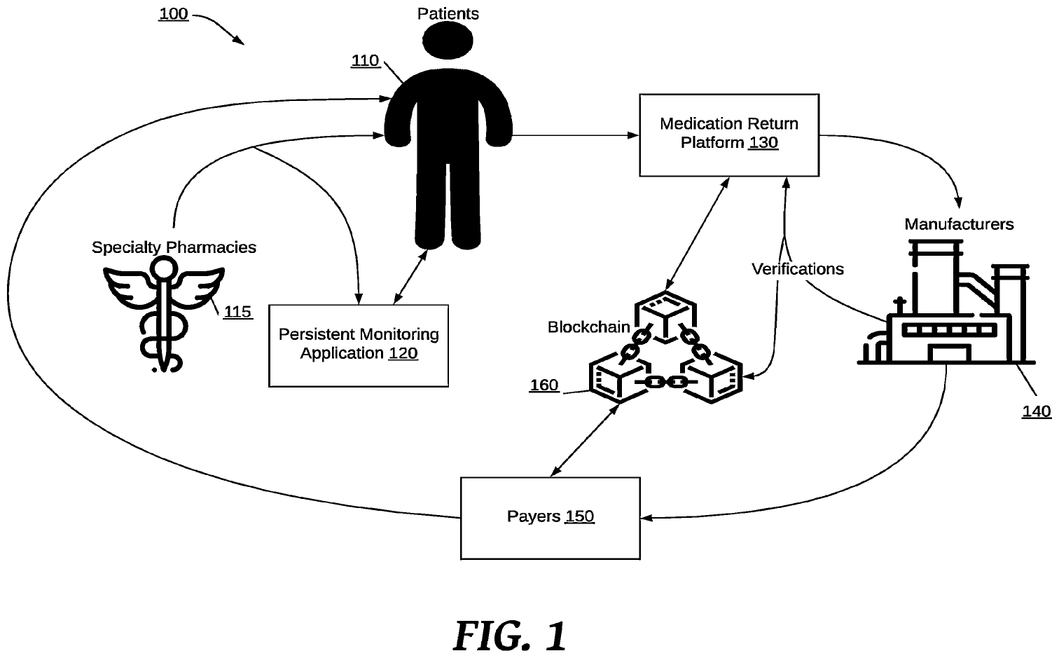

FIG. 1 illustrates an exemplary process for returning unused medications. Patients 110 are prescribed medications by a specialty pharmacy 115. The prescribed medication may be associated with the creation of a persistent monitoring instance of the persistent monitoring application 120. For example, the persistent monitoring instance may be created in a mobile application and or web application that may be accessed by the patient 110 via a network (e.g., the Internet) using a client device (e.g., a smartphone, tablet, laptop, personal computer, and the like). Persistent monitoring instances may be created in the persistent monitoring application 120 for each medication and or patient. Patients 110 may access the persistent monitoring instance to input use data for the prescribed medication into the persistent monitoring application 120. The persistence monitoring application 120 may aggregate use data for medications prescribed to patients 110 to generate forecasts and other predictions using a machine learning system.

Patients 110 may use a medication return platform 130 to return prescribed medications that they do not use. The medication return platform 130 may collect unused medications from patients 110 and return the unused medications to manufacturers 140 for safe disposal. The medication return platform 130 may also document medication returns to generate medication return records that may be stored in a blockchain 160, distributed ledger, and or other secure, public database. Medication return records may include, for example, the medication name, the patient, the amount of medication returned, the return date, and the like.

Manufacturers 140 receive the returned unused medications from the medication return platform 130. Manufacturers 140 include the drug companies that manufactured the returned medications, distributors, specialty medication disposal facilities, and other entities authorized to receive and or destroy unused medications. The manufacturers 140 destroy received unused medications and generate verifications confirming that the returned medications were received and certifying that the returned medications were destroyed and properly disposed of and or recycled. The verifications generated by the manufacturers 140 may be recorded on a blockchain 160, distributed ledger, and or other secure public database.

Rebates for the unused medications purchased by a payer 150 (i.e., the patient 110, the patient's insurance company, the patient's employer, or other insurance provider) may be provided by the manufacturer 140 once the unused medications are returned and or destroyed. Payers 150 may also monitor the verifications and or return records recorded on the blockchain 160 to determine when unused medications purchased by the payers 150 are returned. Payers 150 may request rebates from manufacturers 140 when they determine that a medication purchased by a particular payer 150 is returned.

To facilitate receiving rebates for unused medications, payers 150 may incentivize patients 110 to return their unused medications by distributing de minimis gifts to patients 110 that return unused medications using the medication return platform. De minimis gifts provided by payers 150 may be automatically generated and distributed once a medication return is recorded on the blockchain 160. For example, the de minimis gifts may be non-fungible tokens (NFTs) or other digital currency that may be created and or distributed by a smart contract deployed on the blockchain. The the medication return NFTs may include an NFT smart contract that defines the utility of each medication return NFT. For example, the NFT smart contract may prevent each medication return NFT from being sold and or transferred to another person and or digital wallet. The NFT smart contract may also enable the medication return NFTs to be exchanged for a physical de minimis gift (e.g., a $10 gift card, a certificate of return, and the like). Payers 150 may also provide physical de minimis gifts to patients 110 to incentivize the return of unused medications in lieu of or in addition to the medication return NFTs.

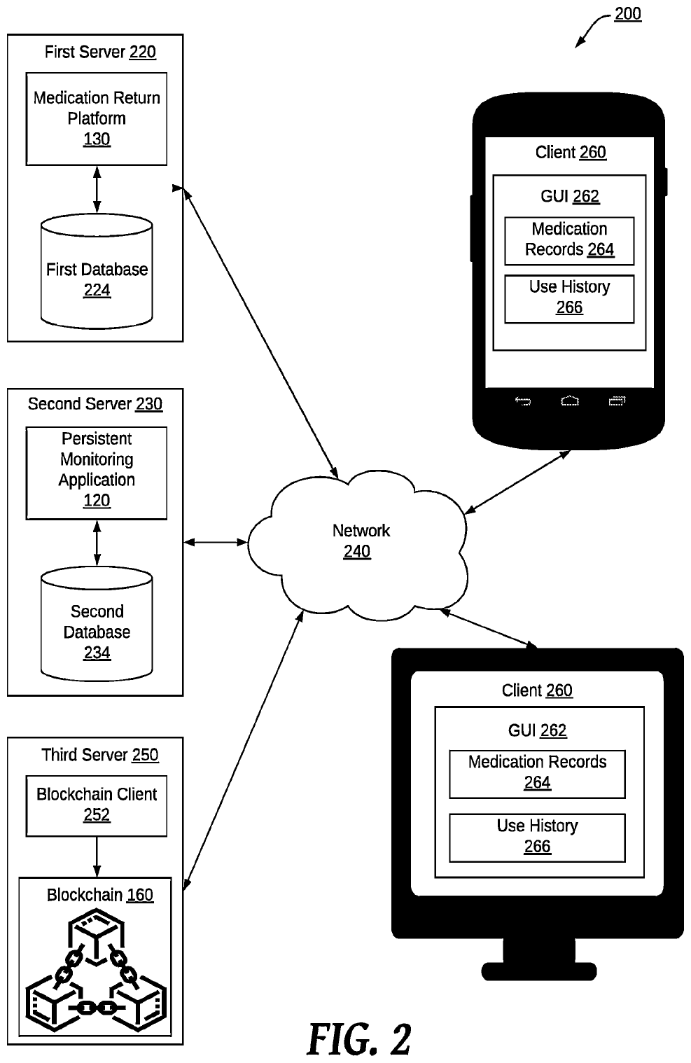

FIG. 2 illustrates a system for returning unused medications according to various embodiments of the present disclosure. System 200 may include a plurality of functional elements that may be provided by mechanical components, electrical components, and or computing devices. The system 200 may implement the medication return process illustrated in FIG. 1. For example, the system 200 may collect unused medications from a patient, verify the amount, authenticity, expiration date, and the like of the collected medications, send notifications to patients, manufacturers, and payers to enable real time tracking of medication returns, verify the receipt and disposal of returned medications, provide rebates for verified medication returns, and provide incentives to patients for returning unused medications. The system 200 may also collect data about patients, disease conditions, therapies, drug distribution logistics, and the like. Data collected by the system 200 may be distributed to manufacturers, payers, healthcare providers, and other industry stakeholders to reduce the amount of wasted medications.

The system 200 may include a first server 220, second server 230, third server 250, and or one or more client devices 260. First server 220, second server 230, third server 250 and or client device(s) 260 are configured to communicate with one another through network 240. For example, communication between the elements can be facilitated by one or more application programming interfaces (APIs). APIs of system 200 can be proprietary and/or can be examples available to those of ordinary skill in the art such as Amazon® Web Services (AWS) APIs or the like. Network 240 refers to the Internet and/or other public or private networks or combinations thereof. Client devices 260 may be any device configured to present graphical user interfaces (GUIs) 262 and receive inputs thereto in the GUIs 262. For example, client device 260 may be a smartphone, personal computer, tablet, laptop computer, or other device.

First server 220 is configured to implement the medication return platform 130 that collects unused medications from patients. To return unused medications, patients may complete medication records 264 that indicate, for example, the medication being returned, the number of doses being returned, the reason for the return, the manufacture date of the medication, the expiration date of the medication, the cost of the medication, and the like. The medication return platform 130 may interface with one or more client devices 260 to provide a GUI 262 for completing medication records 264. The medication return platform 130 may also use data received from medication providers (e.g., medication repository, pharmacy, hospital, drug wholesaler) and or electronic medical records to complete the medication records 264. The medication records 264 for unused medication may be stored in a first database 224. The medication return platform 130 may authorize the return of unused medications based on the medication records 264. Once a return is authorized, the medication return platform 130 may generate a shipping label that patients can use to return unused medications. The medication return platform 130 may also generate a return authorization from that authorizes the manufacturer to destroy the returned medication. Completed return authorization forms may be received from the patient by the medication return platform along with the returned medications

The medication return platform 130 may also analyze shipments of unused medications received from patients to generate one or more verifications. The verifications may be used by manufacturers to determine if a rebate should be given for a shipment of unused medications. For example, the verifications may confirm receipt of shipments of unused medications by the manufacturer, verify the amount of the unused medication received, certify proper disposal of the unused medications, and the like. Once the shipments are verified, the medication return platform 130 may distribute rebates to payers for returned medications. Payers may include patients, healthcare providers, health insurance companies, government agencies, government healthcare systems, employers, and the like. Verifications and other records of the returned medication may be stored in one or more databases 224, 234 and or recorded on a blockchain 160 or other distributed ledger.

Second server 230 is configured to implement a persistent monitoring application 120 that collects use data to monitor medication usage by patients. Use data may include sensor data received from one or more sensors and or manually entered user inputs received from patients. The persistent monitoring application 120 may receive sensor data from wearable devices, wearable sensors, smart medicine bottles, and other devices that may track the use of medications by patients. For example, the persistent monitoring application 120 may receive sensor data that indicates the dose of the medication taken, the date and time the dose was taken, the number of doses used by the patient, the amount of medication in the prescription remaining, and the like. The sensor data may also indicate a patient response to the medication. The persistent monitoring application may also interface with client devices 260 to provide a GUI 262 for entering use history 266. The use history 266 input by the patients may be stored as use data. For example, patients may input records of their usage of a medication (i.e., when the used the medication, the dose taken, the frequency in which the doses were taken, and the like). Patients may also input their response to the medication in the GUI 262. For example, patients may input records of side effects, successful treatment outcomes, unsuccessful treatment outcomes, condition improvements, condition setbacks, and other use history 266 using the GUI 262. Sensor data, use history 266 provided by patients, and other use data may be stored in the second database 234.

Third server 250 may implement a blockchain client 252 that interacts with a blockchain 160 and or other distributed ledger. The blockchain client 252 may record verifications and other data on the blockchain 160 as well as validate blocks, transactions, and other records recorded on the blockchain 160. For example, the blockchain client 252 may interface with the blockchain 160 to publish blocks on the blockchain that record verifications and other records that one or more events included in the medication return process are completed. The return verification blocks may include information that confirms shipments of unused medications are received by manufacturers, certifies the return medications are destroyed, indicates that rebates for returned medications are available and or have been sent, and the like. The blockchain client 252 may also create and manage blockchain accounts, send and receive transactions to and from blockchain accounts, deploy smart contracts and other self-executing code to the blockchain, and read the blocks to extract verifications and other data recorded on the blockchain. The blockchain client 252 may also interface with a blockchain virtual machine to enable smart contracts to run algorithmic calculations, access reference information needed for smart contract execution, withdraw and deposit digital currency from digital wallets, and perform other functions required for smart contact execution. For example, the blockchain client 252 may interface with a blockchain virtual machine to deploy a smart contract that creates and distributes NFTs to patients that return medications and or payers that receive a rebate for the returned medications. The blockchain client 252 may also interact with the blockchain 160 publish data and or public keys, private keys, cloud storage endpoints, and other information for accessing data stored on the first database 224 and or the second database 234 (e.g., medication records 264, use data, and the like) so that this information can be accessed by one or more machine learning systems for further analysis.

First server 220, second server 230, third server 250, first databases 224, second database 234, blockchain 160, and client device(s) 260 are each depicted as single devices for ease of illustration, but those of ordinary skill in the art will appreciate first server 220, second server 230, third server 250, first databases 224, second database 234, blockchain 160, and client device(s) 260 can be embodied in different forms for different implementations. For example, any or each of the first server 220, second server 230, and third server 250 can include a plurality of servers or one or more of the databases 224, 234 or blockchains 160. Alternatively, the operations performed by any or each of first server 220, second server 230, and third server 250 can be performed on fewer (e.g., one or two) servers. In another example, a plurality of client devices 260 communicate with first server 220 second server 230, and or third server 250. A single user can have multiple client devices 260, and/or there can be multiple users each having their own client device(s) 260.

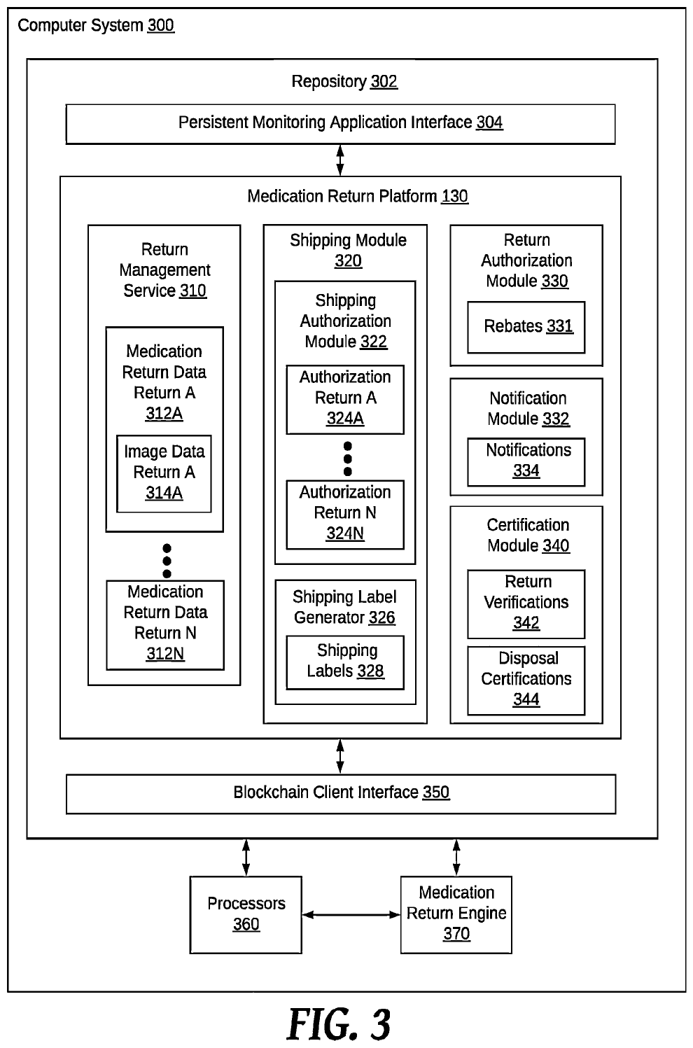

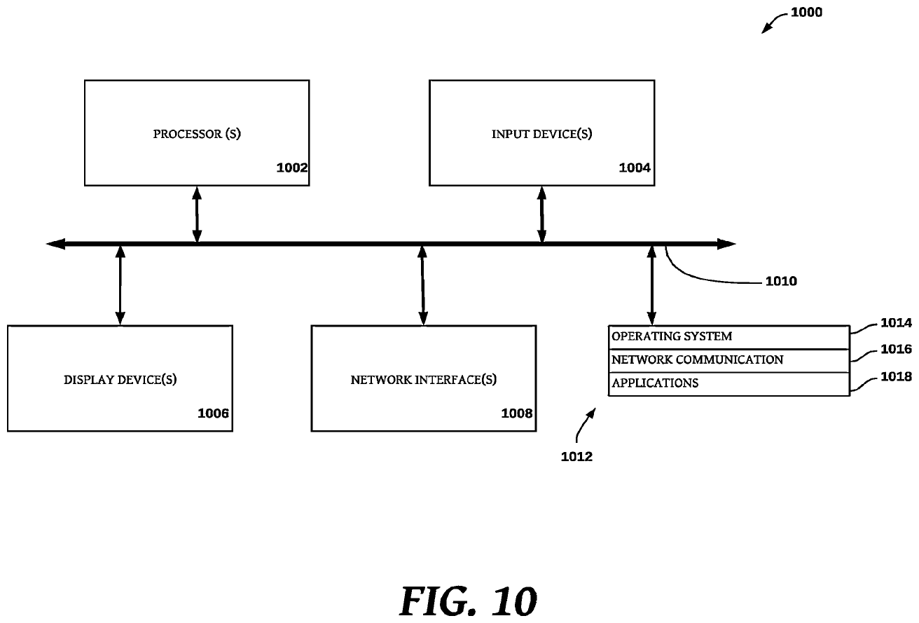

FIG. 3 is a block diagram illustrating an exemplary computer system 300 in accordance with one or more embodiments disclosed herein. The computer system 300 includes a repository 302, a medication return engine 370, and one or more computer processors 360. In one or more embodiments, the computer system 300 takes the form of the computing device 1000 described in FIG. 10 in the accompanying description below or takes the form of the client device 260 described in FIG. 2. In one or more embodiments, each computer processor 360 takes the form of the computer processor 1002 described with respect to FIG. 10.

In one or more embodiments, the repository 302 is any type of storage unit and/or device (e.g., a file system, database, collection of tables, or any other storage mechanism) for storing data. Further, the repository 302 can include multiple different storage units and/or devices. In one or more embodiments, the repository 302 includes a persistent monitoring application interface 304, the medication return platform 130, and a blockchain client interface 350.

The medication return platform 130 includes a return management service 310 that collects medication return data 312A, . . . , 312N required to process requests for returns of unused medications. Medication return data 312A, . . . , 312N may include medication records collected by the medication return platform 130. For example, the medication return data 312A, . . . ,312N may include patient information (e.g., the condition treated by the medication, the date the patient started experiencing symptoms, the patient's symptoms, the number of different medications prescribed to the patient, and the like), insurance information, treatment history, a description of the medication being returned (e.g. the name of the medication, the amount of medication being returned, the expiration date of the medication, the manufacture date of the medication, the manufacturer of the medication, and the like), and other medical information and/or medication information. Patient information may also include identification information (e.g., user name, password, security pin, account information, and the like) for a patient's user account on the medication return platform 130. Patient information may also include personal identification information (e.g., name, address, city, state, zip code, email address, date of birth, social security number, and the like) and other patient information collected by patient history GUIs including questionnaires for eliciting patient information. Patient information may also include a condition history about one or more conditions associated with the medication returned by the patient. Condition history may include the date the patient was diagnosed with the condition, the reason the patient is switching treatments, the date the patient started the treatment she is returning, previous treatments used by the patient, the side effects and/or benefits of using the previous treatments, the date the patient first experienced symptoms related to her condition, the date the patient experienced a recent setback associated with her condition, the treatments the patient has been exposed to, and the like. Insurance information may include the provider name, the name of policy holder, policy number, group number, date of birth of the patient and or policy holder, pharmacy name, and any other information required to identify the insurance policy of the patient. Treatment history may include records of previous drugs and other treatments used by the patient for a particular condition. For example, treatment history may include the medical condition associated with a prescribed treatment, the name (e.g., brand name and/or chemical name) of the treatment the patient would like to return, the side effects and benefits of the treatment, and the like. The description of the medication being returned may include the quantity of pills, capsules, syringes, and/or other medication units the patient is returning, the lot number of the medication, the batch number of the medication, the expiration date of the medication, and the like.

Medication return data 312A, . . . , 312N may also include use data (i.e., sensor data and user inputs) received from the persistent monitoring application. For example, the return management service 310 may use the persistent monitoring application interface 304 (e.g., an API or other communications interface) to request and receive use data from the persistent monitoring application. Each piece of medication return data 312A for a particular medication return (i.e., return A) may include image data 314A of the returned medications. For example, the image data 314A may include a picture of the medication being returned, a picture of the label of the medicine bottle or other container storing the medication, a picture of the purchase receipt for the medication, and the like.

The return management service 310 may automatically extract medication return data 312A, . . . , 312N from an electronic medical record associated with the patient and or the image data 314A. For example, the return management service 310 may include an optical character recognition (OCR) application, a computer vision application, or other image processing service for extracting text and images from the image data 314A. The return management service 310 may also interface with an electronic medical records system to retrieve medical records for the patient submitting the return and extract medication return data 312A, . . . , 312N from the patient's records. The return management service 310 may also receive medication return data 312A, . . . , 312N collected using one or more GUIs included in the medication return platform 130 and or persistent monitoring application. For example, condition history (i.e., patient symptoms, diagnosis data, treatments tried, effects of each treatment, and the like) may be collected using one or more condition history GUIs including questionnaires for eliciting condition history information from patients.

Medication return data 312A, . . . , 312N may be transmitted to the shipping module 320 for review by the shipping authorization module 322. The shipping module 320 may also include a shipping label generator 326 that generates shipping labels 328 for medication returns that are authorized for shipping by the shipping authorization module 320. To authorize the medication returns for shipping, the shipping authorization module 320 may review the medication return data 312A, . . . , 312N for each return to determine if the medication can be returned. For example, the shipping authorization module 320 may verify the patient's identity, the condition of the returned medication, if the returned medication can be accepted by the manufacturer, and the like. To verify the condition of the returned medication, shipping authorization module 322 may review image data of the unused medication. For example, the shipping authorization module 322 may review a photo or other medication image data 220 using an image processing service. The image processing service may include object recognition and or image classification functions that enable the shipping authorization module 322 to determine the condition of the unused medication (i.e., new, used, partially used, sealed packaging, open packaging, and the like) and confirm the accuracy of the medication description included in the medication return data 312A, . . . , 312N (i.e., count the quantity of medication being returned, determine the expiration date, identify manufacturer, and the like).

To verify the condition and description of the returned medication, the shipping authorization module 322 may compare the medication description to the medication image data using the image processing service. The shipping authorization 322 module may use the image processing service to extract one or more fields of data from the medication image data 314A using one or more of optical character recognition (OCR) techniques, machine learning based entity extraction techniques, computer vision based object recognition techniques, computer vision based image feature extraction techniques, machine learning based image classification techniques, and or other automated image processing techniques. For example, the shipping authorization module 322 may use the image processing service to recognize a distinct font on the medication bottle and or bar codes, QR codes, color scheme or distinct hex colors that appear on the medication and or medication packaging shown in the medication image data 314A. The shipping authorization module 322 may also determine the condition of the medication using a machine learning based image classification algorithm. The shipping authorization module 322 may also count the number of doses of medication being returned shown in the image data of the unused medication using a computer vision-based image segmentation and or object recognition algorithm. To determine if a return is authorized for shipping, the shipping authorization module 322 compares the condition and description of the medication determined from the medication image data 314A to the medication description included in the medication return data 312A.

The shipping authorization module 322 may also process the LOT numbers and corresponding expiration dates extracted from the medication image data 314A to confirm with the drug manufacturer that the returned medication is authentic. For example, the shipping authorization module 322 may look up the LOT numbers and or expiration dates for unused medication in a database of authentic drugs provided by the drug manufacturer or other entity. The shipping authorization module 322 may also verify the condition of the medication and the medication description by searching the medication description against one or more databases of medication information.

Once the quantity, condition, expiration information, and or other medication return data 312A, . . . , 312N for the unused medication is verified, the shipping authorization module 322 may verify the identity of the patient, and or determine if the medication can be accepted by the manufacturer. The shipping authorization module 322 may determine if the medication can be accepted by comparing the verified medication description to one or more pre-defined criteria set by the manufacturer and or regulators. For example, the pre-defined criteria may require shipments of unused medication to include a minimum number of units, a treatment for a certain type of condition, a specific brand name or chemical class of treatment, a particular drug manufacturer, a certain insurance provider, a minimum condition (i.e., good, in sealed packaging, unused and the like), an expiration date that is expired, an expiration date that expired within a certain period of time, an expiration date that is not expired, a manufacture date within a certain period of time, a location within a certain distance from the manufacturer, and the like.

The shipping authorization module 322 may also verify the identity of the patient and the medication description and or determine whether to accept the medication based on authorization information received from a provider, manufacturer, and or payer. To obtain authorization information, the shipping authorization module 322 may transmit medication return data 312A, . . . , 312N including, for example, the medication description, patient information, insurance information, treatment history, and/or condition history to a provider (e.g., a healthcare provider, health insurance company, pharmacy, pharmaceutical manufacturer, pharmacy benefit manager, government agency, government healthcare system, and the like), manufacturer, and or payer. The shipping authorization module 322 may then receive authorization information for the patient and/or medication from the healthcare provider, manufacturer, and or payer. For example, authorization information may include LOT numbers and corresponding expiration dates received from drug manufacturers. Authorization information may also include prescription information that verifies the patient was prescribed the unused medication and or received the unused medication from a pharmacy. Authorization information may also include the cost of the medication to the patient, the patient's health insurance company, the patient's employer, and or other payer. Authorization information can also include dates the patient started treatment and other medical information (e.g., treatment history data, condition history, and the like) received from the patient's electronic medical records and or the patient's physician or other healthcare provider.

The shipping authorization module 322 may use the authorization information to verify the patient's identity and or medication description by matching the received authorization information with the medication return data 312A, . . . , 312N. For example, the shipping authorization module 322 may match the patient name and/or prescription date pictured on the medication label and or included in the medication description with the patient name and prescription date included in prescription information obtained from the patient's health insurance company and/or pharmacy. The shipping authorization module 322 may also match the LOT number and expiration dates included in image data 314A of the unused medication (i.e., an image of the label on a medication bottle or package) with the LOT number and expiration date received from the drug manufacturer. The shipping authorization module 322 may also match the patient's date of diagnosis and starting date of treatment with the patient's treatment history recorded in an electronic medical record received form the patient's healthcare provider.

The shipping authorization module 322 generates shipping authorizations 324A, . . . ,324N for return requests that are authorized using one or more of the review and or authorization processes described above. Return requests that are not authorized by the shipping authorization module 322 are marked as unauthorized and the shipping authorization module 322 and or notification module 332 may generate a notification that informs the patient that the return was not accepted and indicates any deficiencies that prevented the return from being authorized. A blockchain client interface 350 may record shipping authorizations 324A, . . . , 324N and or unauthorized shipments on a blockchain to enable entities to track new shipments and rejected shipments by reading the blockchain.

Shipping authorizations 324A, . . . , 324N generated by the shipping authorization module 322 are transmitted to the shipping label generator 326. The shipping label generator 326 generates a shipping label 328 for each return shipment that is authorized by the shipping authorization module 322. Shipping labels 328 generated by the shipping label generator 328 may ship retuned medications directly to the manufacturer of the unused medication and or a centralized clearing house that receives shipments from multiple manufacturers. The shipping label generator 326 may use medication return data 312A, . . . , 312N to generate the shipping labels 328. For example, to generate the shipping labels 328, the shipping label generator 326 may read medication return data 312A, . . . , 312N associated with each authorized return to extract the patient's name, address, city, state, zip, and other shipping information from the patient information. The shipping label generator 326 may then automatically populate the patient's return address into the shipping labels 328. The shipping label generator 326 may also extract the name, address, city, state, zip, and other shipping information for the manufacturer of the unused medication from the medication description to populate the receiving party information into the shipping labels 328.

The shipping label generator 326 may also embed additional medication return data 312A, . . . , 312N into the shipping labels 328. To embed additional medication return data 312A, . . . , 312N, the shipping label generator 326 may generate a QR code, bar code, or other form of machine readable information containing medication return data 312A, . . . , 312N. The code including embedded medication return data 312A, . . . , 312N may then be added to the shipping labels 328 and/or added separately to the medication shipment. Embedded medication return data 312A, . . . , 312N may facilitate processing of the medication shipment once it is received from the patient. For example, embedded manufacturing information, ingredients, disposal instructions, and other medication information extracted from the medication description may facilitate properly destroying the unused medication. Embedded cost information and insurance information may facilitate sending a rebate to the payers of the returned medications.

The medication return platform 130 may also include a return authorization module 330 that reviews the authorized shipments of unused medication received by the medication return platform 130. The return authorization module 330 may review shipments to determine if a rebate for the unused mediations can be provided to payers. The return authorization module 330 may also calculate the amount of the rebate according to the amount of unused medication returned. Medication shipments received by medication return platform 130 may include medication return data 312A, . . . , 312N, insurance information, and the like as well as the physical returned medication. The return authorization module 330 may analyze the received pieces of data and/or the returned medication to determine whether to accept the medication shipment. Based on the analysis performed by the return authorization module 330, the medication return platform 130 may accept or reject a medication shipment. For example, if the return authorization module 330 determines the returned medication meets one or more pre-defined criteria of the manufacturer (e.g., includes a minimum number of units, a treatment for a certain type of condition, a specific brand name or chemical class of treatment, a certain insurance provider, a minimum condition (i.e., good, in sealed packaging, unused and the like), an expiration date that is expired, an expiration date that expired within a certain period of time, an expiration date that is not expired, a manufacture date within a certain period of time, and the like, the return authorization module 330 may accept the return and or provide a rebate for the returned medication. If the return authorization module 330 determines the returned medication does not meet one or more of the pre-defined criteria, the return authorization module 330 may reject the medication shipment and or not provide a rebate for the returned medication.

The return authorization module 330 may determine to accept or reject a medication shipment based on an analysis of the image data 314A for the medication. For example, the return authorization module 330 may compare the medication image data 314A to image data capturing the physical retuned medication. The return authorization module 330 may include an image processing service that may have a camera configured to capture image data of the physical returned medication. The image processing service may also include one or more computer vision and or machine learning algorithms that may extract and or recognize a distinct font on the medication bottle and/or bar codes, QR codes, color scheme or distinct hex colors and or other features that appear in the image data of the physical returned medication and/or medication packaging. The return authorization module 330 may also extract the LOT numbers and corresponding expiration dates and other features of the returned medication from the image data of the physical returned medication to confirm with the drug manufacturer that the returned medication is authentic. Returned medication that matches the medication image data 314A for the unused medication in the shipment or is otherwise determined to be authentic by the return authorization module 330 (e.g., the image data of the physical returned medication includes one or more distinct features of authentic drugs, the physical returned medication meets one or more of the pre-defined criteria, and the like) may be accepted. Returned medications may be rejected if the return authorization module 330 is unable to authenticate the returned medications.

For accepted medications, the return authorization module 330 may generate rebates 331 that reimburse payers for the cost of the returned medications. The accepted medications are returned to the manufacturer for disposal and or are otherwise destroyed to remove them from circulation and ensure that the medications are safely disposed of and are not released into the environments as pollution. The return authorization module 330 may calculate the amount for the rebates 331 based on the cost of the medication, the amount of medication returned, and or a pre-defined rebate rate agreed upon by the manufacturer and the payer. The rebate rate may be specific for each type of medication and may be different for each manufacturer and or each payer. The rebate rate and or the ability of the medication return platform to provide a rebate may depend on the warranty status of the returned medication. For example, the warranty status of the returned medication may include whether the medication is on warranty (i.e., is returned before the warranty expires) or is off warranty (i.e., is returned after the warranty expires). The medication description information may include the warranty period (i.e., 0 days, 90 days, 2 years, and or any other warranty period) as well as the warranty expiration date. The return authorization module 330 may review the warranty period and or the warranty expiration date included in the medication description information to determine if a rebate is authorized and if so calculate the rebate rate and the rebate amount based on the, warranty period, warranty expiration date, and or warranty status of the returned medication The rebate rate may be determined automatically based on previously calculated rebate rates for other medications. For example, the rebate rate may be determined using a k-nearest neighbors (K-NN) algorithm or other machine learning based classification algorithm trained using a training dataset including one or more of data fields (i.e., the manufacturer, payer, medication name, condition treated, medication cost, rebate rate for medications, and the like) for medications that are similar to the returned medication. Returned medications that rejected by the return authorization module 330 may still be sent to the manufacture and or destroyed to remove the medications from circulation and ensure safe disposal. However, the return authorization module 330 may not generate rebates 331 for rejected medications.

The medication return platform 130 may also include a notification module 332 that generates notifications 334 used to track medication returns. The notifications 334 generated by the notification module 332 may include user notifications and/or entity notifications. The notifications 334 may be distributed to users and entities (i.e., manufacturers, healthcare providers, payers, and the like) as electronic messages (i.e., push notifications, email messages, and the like). The notifications 334 may also be recorded on a blockchain using the blockchain client interface 350. User notifications may update patients on the status of their returned medications. For example, user notifications may be distributed to patients when a shipment is authorized, a return is received, a shipment is authorized, a rebate is generated and the like. User notifications may also include status updates from the shipping module 320 and or the return authorization module 330, for example, notifications 334 that the medication return data for the medication return been received, that the medication return data for the medication return has been verified, that the medication return data is incorrect or incomplete, that the shipping label has been generated, that the returned medication has been authenticated and the like. User notifications may be distributed to patients and other users of the medication return platform 130 in real time as push notifications or other notifications delivered to a client device that may be displayed in a GUI.

Entity notifications may be distributed to healthcare providers, pharmacies, insurance companies, drug manufacturers, and the like to help entities adjust to changes in the number of patients using a particular treatment. The notifications may include updates on the status of returns as well as treatment history, condition history, and other medication return data 312A, . . . , 312N received from patients. Entities may use the data included in notifications 334 to help predict effective treatments for patients, develop more effective medications, identify the benefits and side effects of a particular treatment, budget for drug costs, streamline drug manufacturing supply chains, manage drug inventories, determine the effectiveness of a particular treatment, and the like. Entity notifications generated by the notification module 332 may also include notices sent to a doctor or other healthcare provider. For example, notifications that a patient is no longer taking a returned medication or other updates to a patient's treatment plan. Entity notifications may also be sent to insurance companies drug manufacturers, and/or other subscribers or providers. For example, notifications that a drug has been returned may be sent to a drug manufacturer to help anticipate the amount of supply needed to satisfy patient demand. Notifications that a patient is no longer taking a returned medication may be sent to an insurance company to update patient coverage information and help insurance companies develop budgets for costs associated with providing pharmaceutical drug coverage. Data included in the notifications 334 may be distributed to patients and or entities and or recorded on the blockchain in real time. Data included in the notifications 334 may also be aggregated by the medication return platform 130 and used to assemble training datasets used to train machine learning models that predict the probability of medication discontinuation for a particular patient and or medication.

The medication return platform 130 may also include a certification module 340. The certification module 340 may generate verifications and or certifications that confirm one or more steps in the medication return process are completed. For example, the certification module 340 may generate return verifications 342 that confirm shipments of unused medications are received by the manufacturers. The certification module 340 may generate the return verifications upon receiving a delivery confirmation that the returned medications have been delivered to the manufacturer. For example, the certification module 340 may extract the delivery confirmation from tracking data for the shipment of unused medication provided by the carrier (i.e., UPS®, FedEx®, USPS®, and the like). The manufacturer may also provide a delivery confirmation directly to the medication return platform 130. For example, the manufacturer may send a message to the medication return platform 130 confirming the shipment of unused medication was received. The manufacturer may also maintain a database of returned medication (i.e., a blockchain, distributed ledger, private cloud database, public cloud database, and the like) that may be read by the certification module 340. The certification module 340 may lookup returned medication by LOT number, expiration date, and other medication indication information and retrieve a delivery confirmation for the returned medication when the medication is received by the manufacturer and added to the database.

The certification module 340 may also generate disposal certifications 344 that confirm the returned medications are destroyed and properly disposed. The certification module 340 may generate the disposal certifications 344 upon receiving a disposal confirmation from the manufacturer and or drug disposal facility that the retuned medications are destroyed. For example, the disposal confirmation may be a message from the manufacturer or disposal facility that includes a record of the disposal of the returned medications. The certification module 340 may also retrieve the disposal confirmation from a database maintained by the manufacturer and or drug disposal facility that includes disposal records for the returned medications. To obtain the disposal records, the certification module 340 may search the disposal records in the database by LOT number, expiration date, and or other medication indentation information for a particular returned medication. Return verifications 342, disposal certifications 344 and other confirmations generated by the certification module 340 may be recorded on a blockchain by the blockchain client interface 350. Payers may read the return verifications 342, disposal certifications 344 and other verifications stored on the blockchain to track the status of returned medications and or determine when to request a rebate from the manufacturer. For example, payers may automatically request rebates for all returned medications that have return verifications 342 and or disposal certifications 344 recorded on the blockchain.

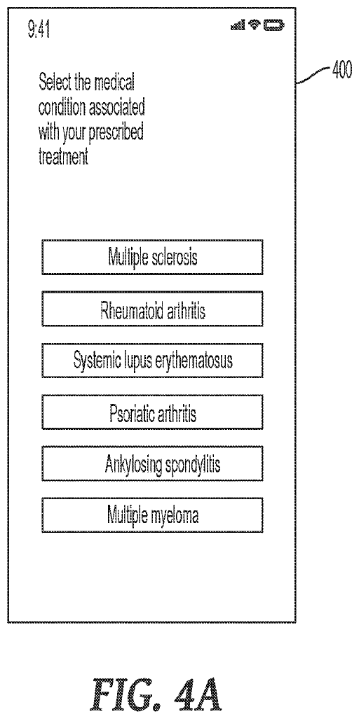

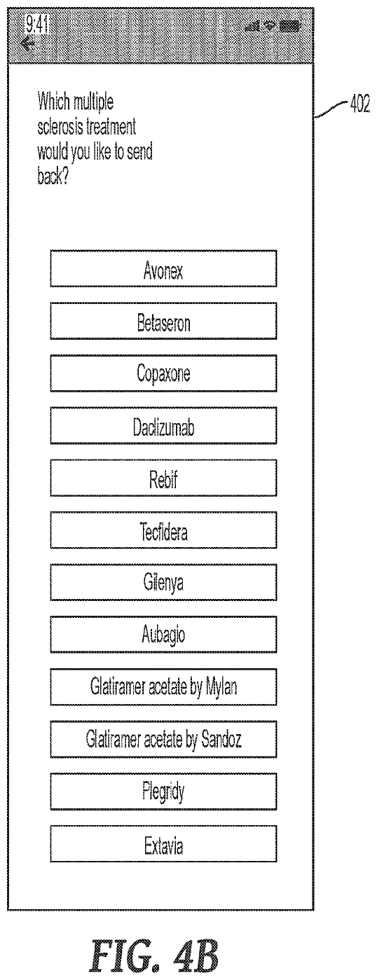

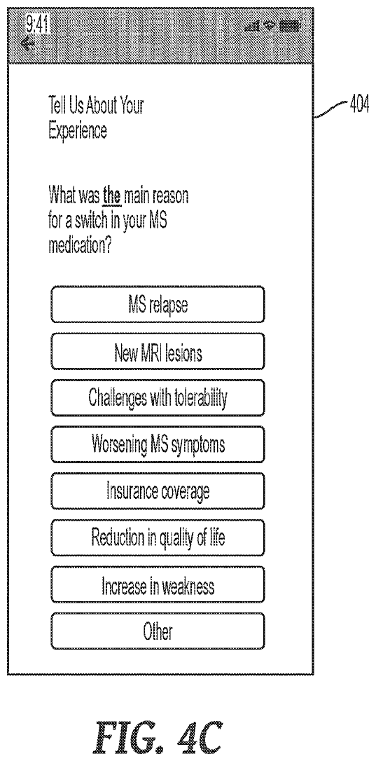

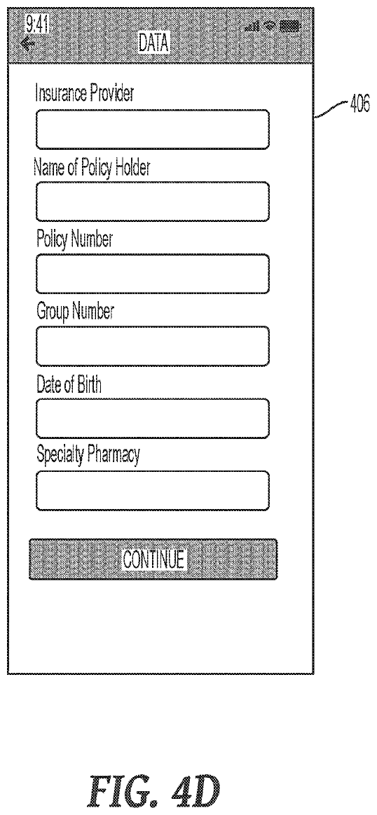

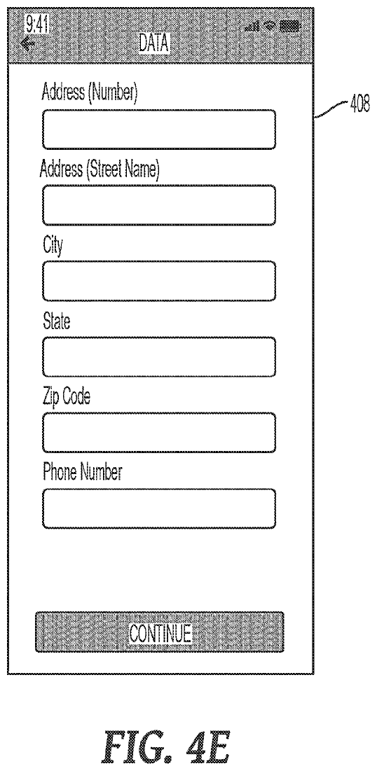

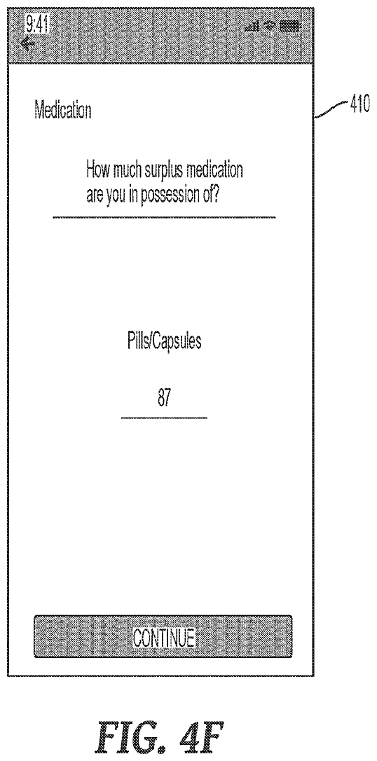

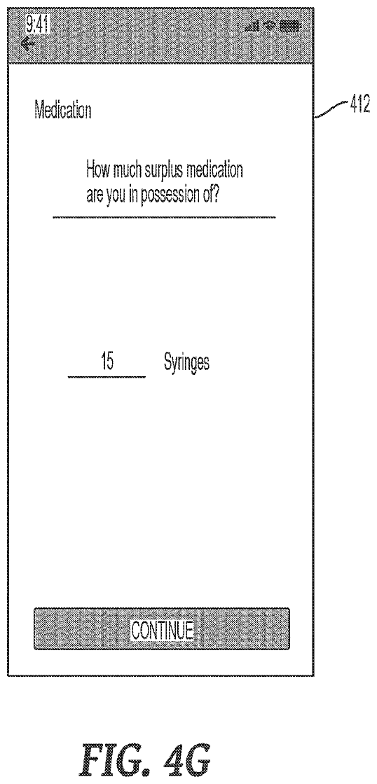

FIGS. 4A-G illustrate exemplary GUIs for collecting user data that may be provided by the medication return platform. FIGS. 4A-C illustrate exemplary GUIs that elicit treatment history information from patients using the medication return platform. Patients and other users may enter treatment history information into the GUIs by, for example, selecting one of the options shown or selecting other and entering text into a free form text box. FIG. 4A illustrates an exemplary medical condition GUI 400 for patients to enter a medication condition associated with the medication they are returning. FIG. 4B illustrates an exemplary medication identification GUI 402 for patients to enter one or more medications they are returning. FIG. 4C illustrates an exemplary treatment problem identification GUI 404 for patients to enter one or more side effects they experienced while taking the medication they are returning. Patients may select and/or enter one or more conditions, medications, and/or side effects in the treatment history GUIs 400-404.

The treatment history GUIs may be displayed sequentially by the medication return platform. For example, the medication return platform may first display a medication condition GUI 400 and receive one or more conditions from the patient. Based on the conditions selected by the patient, the medication return platform may generate a medication identification GUI 402 including medications for the conditions entered by the patient. After receiving the medications from the patient, the medication return platform may then generate a treatment problem identification GUI 404 including the most common problems with the medications entered by the patient and/or the most common reasons for discontinuing use of the identified medications. Treatment history data collected by the treatment history GUIs 400-404 may be stored by the medication return platform and/or distributed to one or more entity participants in to the medication return platform including healthcare providers, drug manufacturers, insurance companies, government agencies, government healthcare systems, and other drug industry stakeholders. Treatment history data may be used to better understand the benefits and problems of using particular medications, predict treatments that will be effective for patients, and the like.

FIGS. 4D-4G illustrate additional exemplary GUIs the elicit insurance, personal, and medication information from patients that may be provided by the medication return platform. Patients and other users may enter insurance information, patient information, medication description information, and other medication return data into the GUIs by, for example, selecting one of the options shown or selecting other and entering text into a free form text box. FIG. 4D illustrates an exemplary insurance information GUI 406 for patients to enter their insurance information. Once received by the insurance information GUI 406, patient insurance information may be used to determine if the payer is able to receive a rebate for the returned medication, identify the insurance company, employer, or other payer to send the rebate to, calculate the rebate rate for the returned medication, and the like. FIG. 4E illustrates an exemplary personal information GUI 408 for patients to enter their address, contact information, and other personal information. Once received by the personal information GUI, patient personal information may be used to identify patients, generate a personalized shipping label used to return medications, and the like.

FIGS. 4F and 4G illustrate exemplary medication identification GUIs 410-412 provided by the medication return platform. Medication identification GUIs 410-412 may collect medication descriptions and other information used to identify the medications a patient is returning. FIG. 4F illustrates an exemplary oral medication identification GUI 410. Patients may enter the quantity of pills/capsules they are returning, medication expiration information, and other information describing oral medications in the oral medication identification GUI 410. FIG. 4G illustrates an exemplary injection medication identification GUI 412. Patients may enter the number of syringes they are returning, medication expiration information, and other information describing injectable medications in the injection medication identification GUI 412. Medication descriptions received by the medication identification GUIs 410-412 may be used to identify returned medications, determine whether the medication return platform may accept and/or provide rebates for returned medications, and the like.

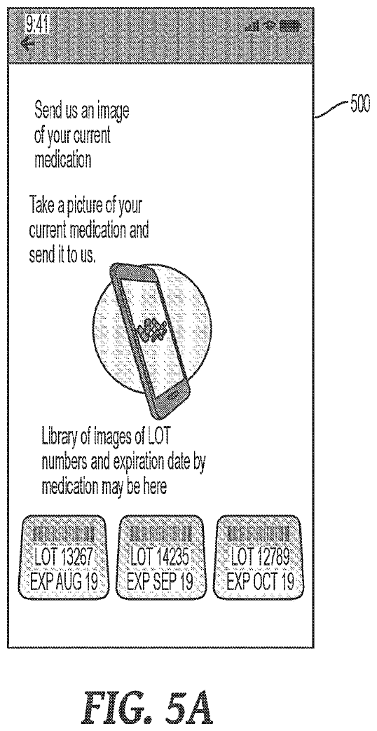

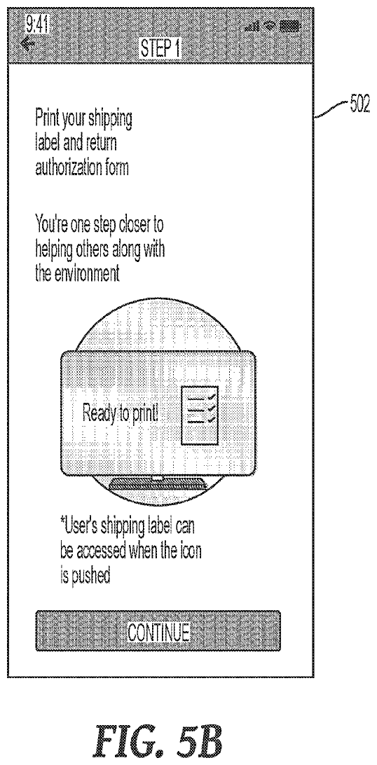

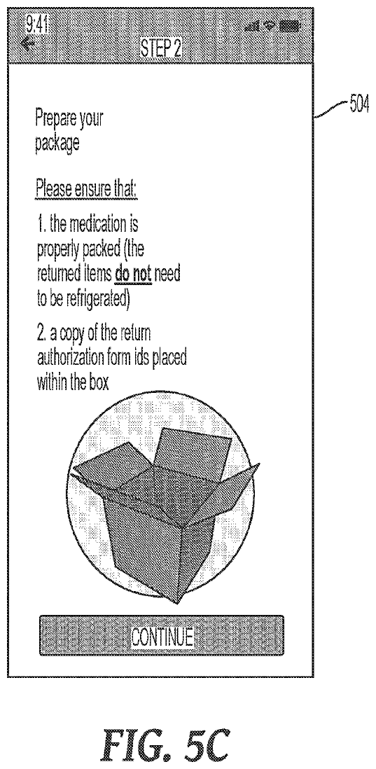

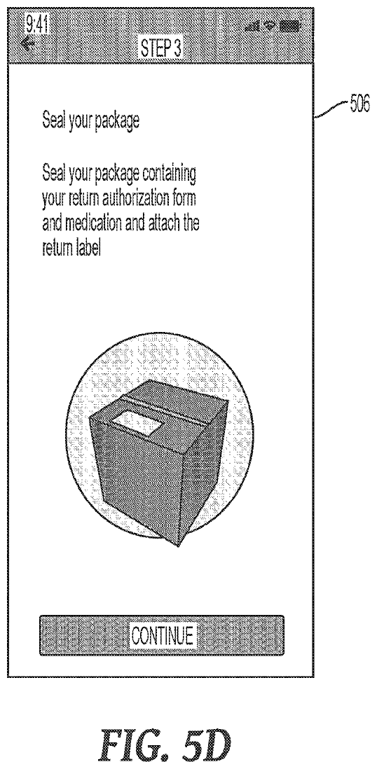

FIGS. 5A-5D illustrate exemplary shipping and packaging GUIs provided by the medication return platform for facilitating packing and shipping returned medications by patients, pharmacies, and other users of the medication return platform. To return medications via the platform, patients and other users may have to follow instructions included in one or more shipping and packaging GUIs. FIG. 5A illustrates an exemplary image capture GUI 500 that facilitates capturing image data of the medication. The image data may include LOT numbers, the expiration date, and other identifying information for the medication returned by the patient. The medication return platform may integrate with a camera included on a client device used by the patient to access the medication return platform. For example, the medication return platform may integrate with a camera included in a smart phone or other mobile device. Once integrated with the camera, the medication return platform may first display the image capture GUI 500. The after a predetermined period of time has passed and/or after prompting by the user, the medication return platform may initialize the camera and display an image capture user interface used to operate the camera. The medication return platform may then receive the image of the medication captured by the user. Previously captured medication images may also be displayed in the image capture GUI 500. Patients may select a previously captured picture to associate it with a shipment of retuned medications.

FIG. 5B illustrates an exemplary shipping label GUI 502. The shipping label GUI 502 may include an icon for accessing a shipping label, instructions for printing a shipping label, instructions for preparing a return authorization form, and the like. Once a medication return has been verified by the verification system, the medication return platform may send a notification to the user that a shipping label for the returned medication is available for printing. The shipping label may be accessed from the shipping label GUI 502. For example, the shipping label may be downloaded by selecting an icon or other prompt on the shipping label GUI 502. The shipping label may also be automatically sent to the patient (e.g., emailed to the patient) in response to the patient selecting an icon or other prompt on the shipping label GUI 502. FIG. 5C and 5D illustrate exemplary packaging GUIs 504, 506 including instructions for packing medication and an authorization form into a medication return package. The authorization form may also be submitted digitally.

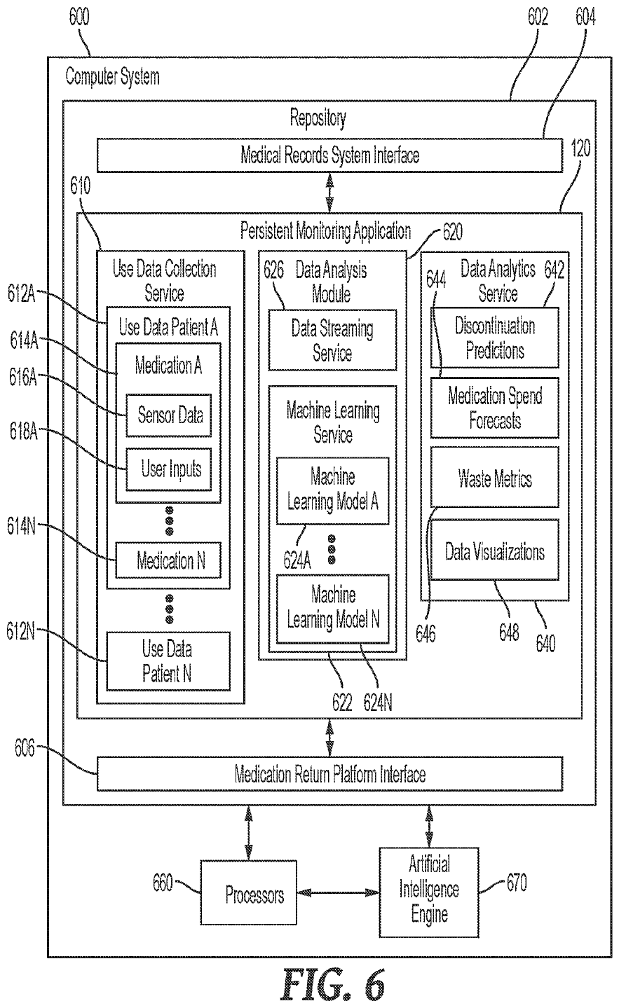

FIG. 6 is a block diagram illustrating an exemplary computer system 600 in accordance with one or more embodiments disclosed herein. The computer system 600 includes a repository 602, an artificial intelligence engine 670, and one or more computer processors 660. In one or more embodiments, the computer system 600 takes the form of the computing device 1000 described in FIG. 10 of the accompanying description below or takes the form of the client device 260 described in FIG. 2. In one or more embodiments, each computer processor 660 takes the form of the computer processor 1002 described with respect to FIG. 10.

In one or more embodiments, the repository 602 is any type of storage unit and/or device (e.g., a file system, database, collection of tables, or any other storage mechanism) for storing data. Further, the repository 602 can include multiple different storage units and/or devices. In one or more embodiments, the repository 602 includes a medical records system interface 604, the persistent monitoring application 120, and a medication return platform interface 606.

The persistent monitoring application 120 is configured to collect use data 612A, . . . , 612N that tracks medication usage by patients. The persistent monitoring application 120 may use sensors (i.e., wearable sensors, smart medicine bottles, smart syringes and other smart medicine delivery devices, and the like) and or user inputs entered in one or more use monitoring GUIs to continuously track the use of medications by patients. For example, the persistent monitoring application 120 may continuously determine use data (e.g., a timestamp indicating when patients take a dose of medication, the amount of medication included in each dose, the amount of medication remaining in a patient's prescription, and the like) for each patient. The persistent monitoring application 120 may determine use data for multiple medications 614A, . . . , 614N prescribed to the patient. A use monitoring instance for each of the medications 614A, . . . , 614N prescribed to the patient may be generated in the persistent monitoring application 120 automatically as soon as the patient receives the prescription.

Each piece of use data 612A for each medication 614A may include sensor data 616A received from one or more sensors and or user inputs 618A received from patients. The persistent monitoring application 120 may receive sensor data 616A from wearable devices, wearable sensors, smart medicine bottles, and other devices that may track the use of medications by patients. For example, the persistent monitoring application 120 may receive sensor data 616A that indicates the dose of the medication taken, a timestamp including the date and time the dose was taken, the number of doses used by the patient, the amount of medication in the prescription remaining, and the like. The sensor data 616A may also indicate a patient response to the medication (i.e., a side effect and or successful treatment outcome experienced by the patient). The persistent monitoring application 120 may also interface with client devices to provide a use monitoring GUIs for entering user inputs 618A that include use data. For example, patients may input records of their usage of a medication (i.e., when they used the medication, the dose taken, the frequency in which the doses were taken, and the like). Patients may also input their response to the medication in one or more of the use monitoring GUIs. For example, patients may input records of side effects, successful treatment outcomes, unsuccessful treatment outcomes, condition improvements, condition setbacks, and other use data in the use monitoring GUIs. Sensor data and other use data provided by patients may be provided to the data analysis module 620 for further analysis.

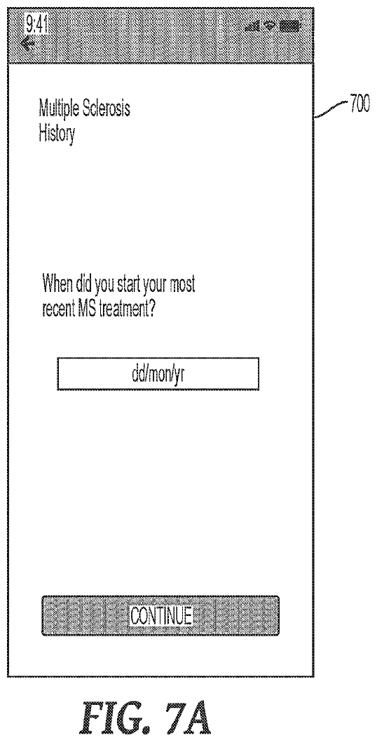

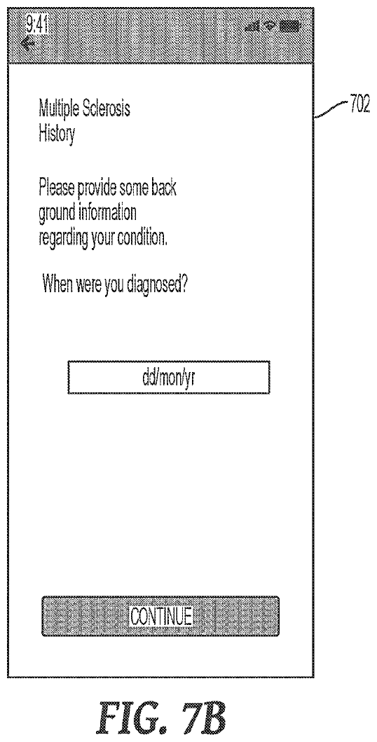

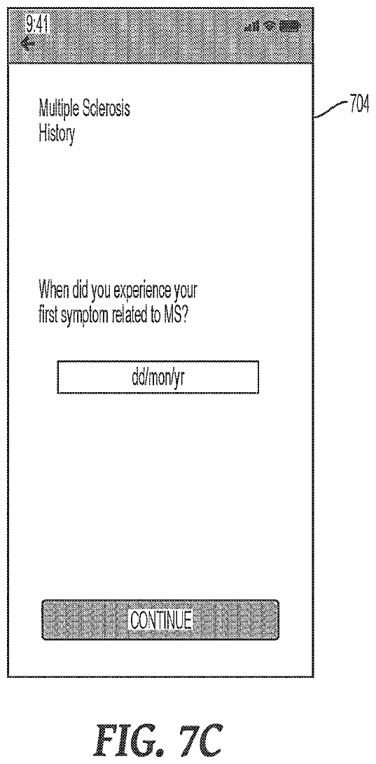

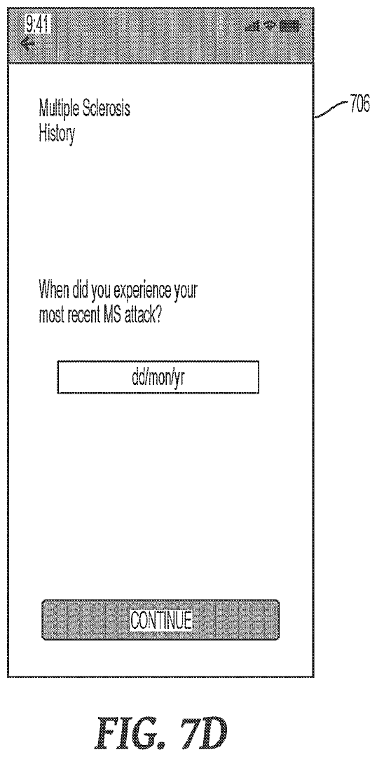

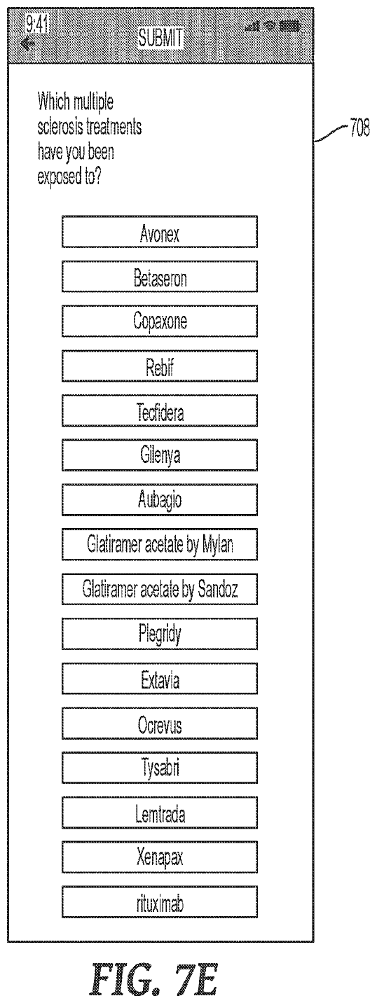

FIGS. 7A-7E illustrate exemplary use monitoring GUIs 700-708 that illicit use data 612A, . . . , 612N from patients. The use monitoring GUIs may also collect medical information including treatment and condition history information from patients using the persistent monitoring application 120. Patients may enter use data 612A, . . . ,612N, condition history, and other medical information into the use monitoring GUIs by, for example, selecting one of the options shown or selecting other and entering text into a free form text box. FIG. 7A illustrates an exemplary diagnosis history GUI 700 for patients to enter condition diagnosis data including diagnosis date. FIG. 7B and 7E illustrate exemplary medication history GUIs 702, 708 for patients to enter medication history data including the start date for their current medication, the names of the medications they have used to treat their condition, and the like. FIGS. 7C and 7D illustrate exemplary symptom history GUIs 704,706 for patients to enter symptom history data including the start date for their first condition symptom, the date of their most recent condition symptom, the type of symptoms, the severity of the symptoms, the duration of the symptoms, and the like. The use monitoring GUIs may also include GUIs for entering use data 612A, . . . ,612N and treatment response data.

Use data 612A, . . . ,612N and other medical information input into the use monitoring GUIs may be transferred to health care providers to update patient medical records. For example, a medical records system interface 604 may connect the persistent monitoring application 120 to an electronic medical records system. The medical records system interface 604 may transfer use data 612A, . . . ,612N and other medical information collected by the persistent monitoring application 120 to the electronic medical records for the patients using persistent monitoring application. The medical records system interface 604 may access the electronic medical records for each patient using the persistent monitoring application 120 and may update the patients medication records with use data 612A, . . . ,612N.

The medical records system interface 604 may also retrieve patient medical data from the electronic medical records system. The persistent monitoring application 120 may also use a medication return platform interface 606 to retrieve medication return data from the medication return platform. The use data 612A, . . . ,612N, patient medical data retrieved from the electronic medical records system, and return medication data received from the medication return platform may be used to generate training datasets that are provided to the data analysis module 620. The data analysis module 620 may include a data streaming service 626 that receives the use data 612A, . . . ,612N, the patient medical data, and or the medication return data and generates training datasets that include portions of the received data. The data streaming service 626 may also preprocess the raw received data before storing the data and or including the data in a training dataset. For example, the data streaming service 626 may clean, transform, and or organize the received data before storing the received data (i.e., use data 612A, . . . ,612N, the patient medical data, and or the medication return data) in a relational database or other storage system.

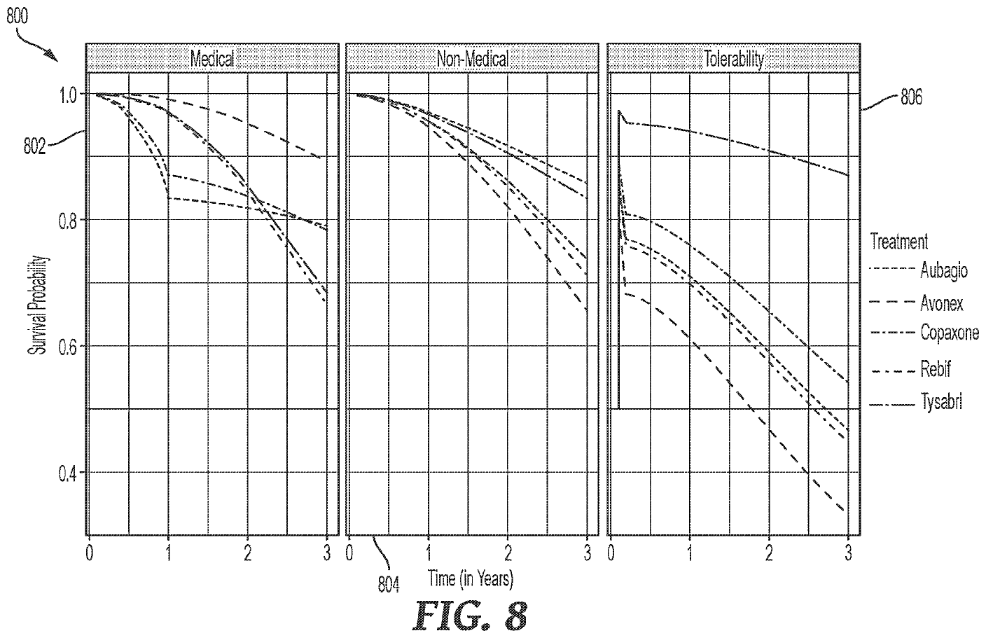

The data streaming service 626 may provide training datasets to the machine learning service 622. The machine learning service 622 may train multiple machine learning models 624A, . . . , 624N using the training datasets provided by the data streaming service 626. A data analysis service 640 may inference one or more of the machine learning models 624A, . . . ,624N to generate outputs (i.e., predictions, forecasts, metrics, visualizes, and the like) that may be used to develop new medications, predict effective treatments, reduce the amount of unused medications, and the like. For example, the machine learning service 622 may use the use data 612A, . . . , 612N, the patient medical data, and or the medication return data to train a machine learning model 624A that predicts a rate of discontinuation for a particular medication. FIG. 8 illustrates an exemplary survival plot 800 showing the discontinuation probability for five different therapies for MS. The survival plot 800 illustrates the survival probability (i.e., the probability a patient will continue using the disease modifying therapies) over a period of three years. The left panel 802 of the survival plot 800 illustrates the survival probability for the five MS therapies based on medical factors including treatment response, adverse reactions, and the like. The center panel 804 of the survival plot 800 illustrates survival probability for the five MS therapies based on non-medical factors including cost, availability, and the like. The right panel 806 of the survival plot 800 illustrates the survival probability for the five MS therapies based on tolerability factors including side effects, amount of effort required to administer the therapy, impact on quality of life, and the like.

The machine learning models 624A, . . . ,624N may be trained using training datasets that include a variety of patient and medication data. For example, the data streaming service 626 may generate training datasets that include demographic information (ethnicity, race, gender, age, weight, prior heath, genetic information, and the like) for a group of patients prescribed a particular mediation to treat a particular condition. The training datasets may also include condition history (i.e., time since the onset of the first symptom of the condition, age at onset, symptoms experienced, and the like) and treatment data (i.e., the number of prior treatments received, the time since the last treatment was stopped, the survival time for each treatment, the name of the prior treatment received, the use data for each treatment, the patient response to each treatment, and the like) for each patient in the group.

To draw insights that are specific to a particular patient, the group of patients included in the training dataset may include patients having similar attributes to the particular patient. Machine learning models 624A, . . . ,624N trained using patient specific data may be patient specific models that more accurately predict the rate of discontinuation for a particular patient or group of patients having one or more attributes in common. The common patient attributes may include, for example, condition history, treatment history, locations, user demographics, or other patient information, and the like. For example, the machine learning service 622 may generate a machine learning model 624A trained using a training dataset including user data for patients taking Avonex that were diagnosed with MS at least 5 years ago. After training, the patient specific machine learning model 624A generated based on this particular training dataset could predict the rate of discontinuation for Avonex for a particular patient that was diagnosed with MS at least 5 years ago. The patient specific machine learning models generated based on user data collected by the persistent monitoring application 120 and or the medication return platform can predict discontinuation rates for specific drugs and specific patients more accurately than models trained on user data that is not specific to a particular patient or group of patients. Over time as more information is collected from users, the machine learning models 624A, . . . ,624N may be re-trained with updated patient data to generate more accurate patient specific models.

The machine learning models 624A, . . . , 624N generated by the machine learning service 622 may have uses beyond personalized medicine. For example, the data analysis service 640 may use the discontinuation predications 642 generated by the machine learning models 624A, . . . , 624N to generate medication spend forecasts 644 that predict the cost of purchasing medications for a group of insured patients over a period of time. Insurance companies may also use the discontinuation predictions 642 to determine which therapies they should cover and purchase from manufacturers. For example, the predicted discontinuation rate included in the discontinuation predictions 642 for a particular drug may be compared to a pre-defined threshold discontinuation rate. If the predicted discontinuation rate is high and above the threshold, the insurance company may determine the treatment is not effective and may not authorize coverage for the treatment. If the predicted discontinuation rate is low and below the threshold, the insurance company may determine the treatment is effective and may authorize coverage and/or prioritize review of the treatment for approval.

Discontinuation predictions 642 for specific medications may also be used by drug manufacturers to develop more effective drugs and better educate prescribers about the medications and expected level of support and or care required to continuing using the therapy. The data analytics service 640 may also use the discontinuation predictions 642 generated by the machine learning models 624A, . . . ,624N to calculate waste metrics 646. For example, the waste metrics 646 may include the amount of unused medications collected by a group of patients over a period of time, the cost of the unused medications, and the like. The data analytics service 640 may also generate data visualizations 648 including charts, graphs, and other diagrams that can be used to illustrate the use data 612A, . . . ,612N, medication return data, and other patient medical information received by the data streaming service 626.

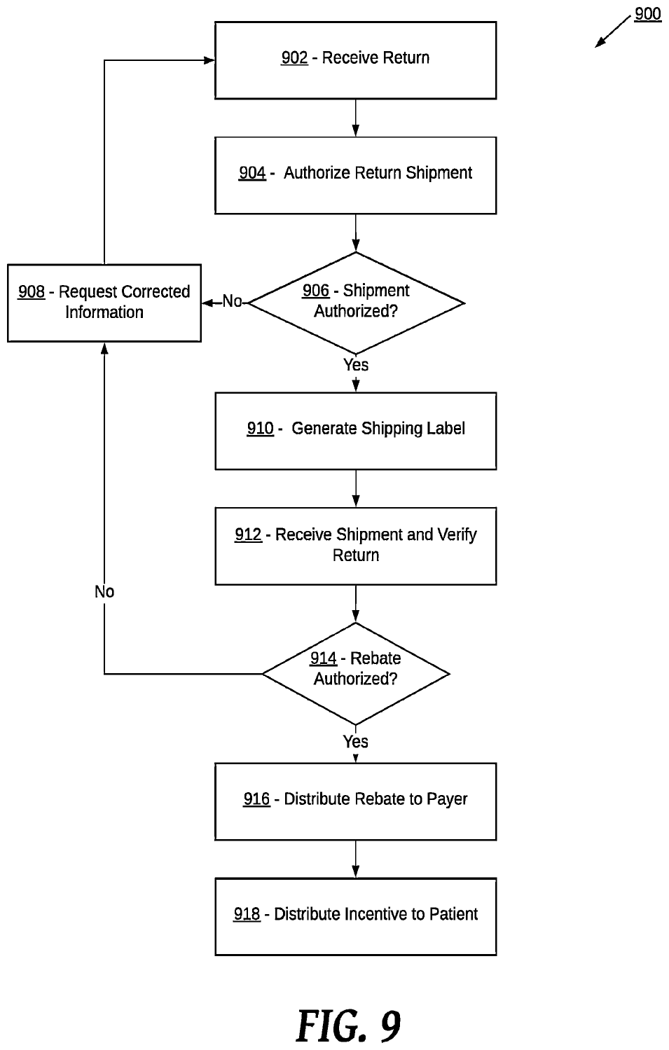

FIG. 9 is a flow chart illustrating an exemplary method for returning unused medication 900. At 902, the medication return platform receives a medication return request including medication return data from a patient. Medication return data may include personal information about the patient, a description of the medication the patient is returning, the patient's condition and treatment history, the patient's insurance information, and the like. The user data may also include image data of the medication the patient is returning, and a digital authorization form signed by the patient that authorizes the medication repository and or manufacturer to accept the returned medication and destroy the medication.

At 904, the medication return platform reviews and the medication return data to authorize a shipment of the returned medication. For example, the medication return platform may confirm the quantity and expiration information of the returned medication provided by the user matches the LOT number and expiration information shown in the medication image data. At 906 if the medication return platform authorizes the shipment of the returned medication by verifying the medication information included in the medication return data, the medication return platform may generate a shipping label for the returned medication. At 906 if the medication return platform does not verify the medication information included in the medication return data, the medication return platform may request that the patient provide corrected and or additional medication return data at 908. For example, the medication return platform may request the patient update the medication quantity and or expiration information. Updated medication return data may then be received at 902 and verified at 904.

At 912, the medication return platform receives the shipment of returned medication from the patient. The shipment may be accepted by a medication repository and or a manufacturer of the returned medication. The physical returned medication and medication return data may be verified at 912 to determine if a rebate for the returned medication is authorized. To determine if a rebate is authorized the verified medication return data from 904 may be compared to the amount of unused medication received in the shipment. For example, the condition of the received medication, quantity of the received medication, authenticity of the received medication, and the like may be compared to the amount of medication described in the verified medication return data. If the received amount of unused medication matches the medication described in the verified medication return data, the return may be accepted and the rebate may be authorized. The purchaser of the medication and the manufacturer of the medication may also be verified to determine if a rebate can be provided for the returned medication. At 914, if the medication return platform verifies the return, a rebate for the returned medication is authorized and the medication return platform calculates the rebate amount. At 914, if the medication return platform determines the return is not verified, the medication return platform may provide a notification indicating the reason(s) that prevented the return from being verified. The notification may be distributed to the patient and may include a request that the patient provide additional and or corrected medication return data at 908. For example, the medication return platform may request the patient update the amount of the medication included in the medication return data and or provide the name and or contact information for the patient's health insurance provider. Updated medication return data may then be received at 902 and the medication return platform may determine if the return is verified and a rebate is authorized at 914.

At 916, the medication return platform may distribute the rebate for the cost of the returned medication to the payer. At 918, the medication return platform may distribute an incentive the patient. The incentive may include, for example, de minimis gifts (e.g., gifts cards of limited value and the like) and or NFTs and other digital currency. The NFTs and other digital currency may have limited functionality provided by smart contracts or other self-executing code deployed on a blockchain. For example, the NFTs may not be transferable or exchangeable for items having a monetary value that exceeds a pre-defined amount.

FIG. 10 shows a computing device according to an embodiment of the present disclosure. For example, computing device 1000 may function as client (which may include a platform for returning an managing unused medications). The computing device 1000 may be implemented on any electronic device that runs software applications derived from compiled instructions, including without limitation personal computers, servers, smart phones, media players, electronic tablets, game consoles, email devices, etc. In some implementations, the computing device 1000 may include one or more processors 1002, one or more input devices 1004, one or more display devices 1006, one or more network interfaces 1008, and one or more computer-readable mediums 1012. Each of these components may be coupled by bus 1010, and in some embodiments, these components may be distributed among multiple physical locations and coupled by a network.

Display device 1006 may be any known display technology, including but not limited to display devices using Liquid Crystal Display (LCD) or Light Emitting Diode (LED) technology. Processor(s) 1002 may use any known processor technology, including but not limited to graphics processors and multi-core processors. Input device 1004 may be any known input device technology, including but not limited to a keyboard (including a virtual keyboard), mouse, track ball, camera, and touch-sensitive pad or display. Bus 1010 may be any known internal or external bus technology, including but not limited to ISA, EISA, PCI, PCI Express, NuBus, USB, Serial ATA or FireWire. Computer-readable medium 1012 may be any medium that participates in providing instructions to processor(s) 1002 for execution, including without limitation, non-volatile storage media (e.g., optical disks, magnetic disks, flash drives, etc.), or volatile media (e.g., SDRAM, ROM, etc.).

Computer-readable medium 1012 may include various instructions 1014 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 1004; sending output to display device 1006; keeping track of files and directories on computer-readable medium 1012; controlling peripheral devices (e.g., disk drives, printers, etc.) which can be controlled directly or through an I/O controller; and managing traffic on bus 1010. Network communications instructions 1016 may establish and maintain network connections (e.g., software for implementing communication protocols, such as TCP/IP, HTTP, Ethernet, telephony, etc.).

Application(s) 1018 may be an application that uses or implements the processes described herein and/or other processes. For example, an shipping module that verifies medication return data and generates shipping labels, a return authorization module that authorizes and distributes rebates for returned medication, and/or a medication return platform that collects patient data and facilitates shipping returned medications to a medication repository and or manufacturer. The processes may also be implemented in operating system 914. For example, application 918 and/or operating system 914 may present GUIs that facilitate return of unused medications and distribute notifications and compensation to patients as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, microcontrollers, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A method of managing medication returns comprising:
providing a medication return platform having a graphical user interface that is remotely accessible by a plurality of users;
receiving, by the medication return platform, medication return data including patient information and medication information for an amount of unused medication from a user included in the plurality of users;
verifying, by the medication return platform, accuracy of the medication return data;
generating, by the medication return platform, a shipping label and a return authorization from for the user;
receiving, from the user, a completed return authorization form and the amount of unused medication sent using the shipping label;
matching, by the medication return platform, the verified medication return data with the amount of unused medication;
calculating, by the medication return platform a rebate amount for the amount of unused medication; and
distributing, by the medication return platform, a rebate for the rebate amount to a payer that purchased the amount of unused medication.

2. The method of claim 1, wherein the patient information includes condition history and treatment history relating to multiple sclerosis, glioblastoma multiforme, Parkinson's disease, rheumatoid arthritis, systemic lupus erythematosus, psoriatic arthritis, ankylosing spondylitis, multiple myeloma or other forms of cancer, neuromyelitis optica spectrum disorder, sleep disorders, cystic fibrosis, hematological malignancies, Alzheimer's disease or other neurodegenerative or neurological conditions, hemophilia, hepatitis, HIV/AIDS, Crohn's disease, ulcerative colitis, solid organ tumors, organ transplants, hereditary amyloidosis, metabolic deficiencies, hormonal deficiencies, pulmonary hypertension, hypercholesterolemia, spinal muscular atrophy, muscular dystrophies, or other muscle disorders.

3. The method of claim 1, wherein the graphical user interface is provided by smartphone, tablet or personal computer.

4. The method of claim 1, wherein the rebate amount is calculated based on a rebate rate and the amount of unused medication.

5. The method of claim 1, comprising distributing a incentive to the user, wherein the incentive includes a de minimis gift.

6. The method of claim 1, wherein the verifying further comprises:
transmitting the medication return data to a provider;
receiving, from the provider, authorization information about the amount of unused medication;
receiving, from the user, image data capturing at least a portion of the amount of unused medication;
extracting a feature of the amount of unused medication from the image data; and
matching the authorization information received from the provider with the feature extracted from the image data and the medication return data.

7. The method of claim 6, wherein the feature extracted from the image data includes at least one of a LOT number and a piece of expiration information.

8. The method of claim 6, wherein the provider is a healthcare provider, health insurance company, pharmacy, pharmaceutical manufacturer, pharmacy benefit manager, government agency, or government healthcare system.

9. The method of claim 1, comprising;
generating a return verification that confirms the amount of unused medication is accepted by a manufacturer;
recording the return verification on a blockchain;
reading the blockchain to locate a block that records the return verification; and
requesting a rebate for the amount of unused mediation based on locating the block that records the return verification.

10. The method of claim 1, further comprising:
receiving use data for a medication prescribed to the user, the use data including sensor data for a group of patients, the sensor data indicating a period of time each patient in the group of patients was taking the medication;
training a machine learning model using the use data and the medication return data; and
obtaining a discontinuation prediction for the medication from the machine learning model, wherein the discontinuation prediction predicts an amount of time that the user will take the medication.

11. The method of claim 10, comprising providing the discontinuation prediction to the payer, wherein the payer is at least one of a health insurance company, employer, government agency, and government healthcare system.

12. A system for managing medication returns, comprising:
a processor and memory connected to each other;
a display connected to the processor; and
a plurality of lines of instructions stored in the memory and executed by the processor that is configured to:
provide, on the display, a medication return platform having a graphical user interface that is remotely accessible by a plurality of users;
receive, by the medication return platform, medication return data including patient information and medication information for an amount of unused medication from a user included in the plurality of users;
verify, by the medication return platform, an accuracy of the medication return data;
generate, by the medication return platform, a shipping label and a return authorization from for the user;
receive, from the user, a completed return authorization form and the amount of unused medication sent using the shipping label;
match, by the medication return platform, the verified medication return data with the amount of unused medication; and
distribute, by the medication return platform, a rebate to a payer that purchased the amount of unused medication.

13. The system of claim 12, wherein the patient information includes condition history and treatment history relating to multiple sclerosis, glioblastoma multiforme, Parkinson's disease, rheumatoid arthritis, systemic lupus erythematosus, psoriatic arthritis, ankylosing spondylitis, multiple myeloma or other forms of cancer, neuromyelitis optica spectrum disorder, sleep disorders, cystic fibrosis, hematological malignancies, Alzheimer's disease or other neurodegenerative or neurological conditions, hemophilia, hepatitis, HIV/AIDS, Crohn's disease, ulcerative colitis, solid organ tumors, organ transplants, hereditary amyloidosis, metabolic deficiencies, hormonal deficiencies, pulmonary hypertension, hypercholesterolemia, spinal muscular atrophy, muscular dystrophies, or other muscle disorders.

14. The system of claim 12, wherein the processor is configured to:
transmit the medication return data to a provider;
receive, from the provider, authorization information about the amount of unused medication;
receive, from the user, image data capturing at least a portion of the amount of unused medication;
extract a feature of the amount of unused medication from the image data; and
match the authorization information received from the provider with the feature extracted from the image data and the medication return data to verify the accuracy of the medication return data.

15. The system of claim 14, wherein the feature of the amount of unused medication extracted from the image data includes at least one of a LOT number and a piece of expiration information.

16. The system of claim 14, wherein the provider is a healthcare provider, health insurance company, pharmacy, pharmaceutical manufacturer, pharmacy benefit manager, government agency, or government healthcare system.

17. The system of claim 12, wherein the processor is configured to:
generate a return verification that confirms the amount of unused medication is accepted by a manufacturer;
record the return verification on a blockchain;
read the blockchain to locate a block that records the return verification; and
request a rebate for the amount of unused medication based on locating the block that records the return verification.

18. The system of claim 12, wherein the processor is configured to:
receive use data for a medication prescribed to the user, the use data including sensor data for a group of patients, the sensor data indicating a period of time each patient in the group of patients was taking the medication;
train a machine learning model using the use data and the medication return data; and
obtain a discontinuation prediction for the medication from the machine learning model, wherein the discontinuation prediction predicts an amount of time that the user will take the medication.

19. The system of claim 18, wherein the processor is configured to provide the discontinuation prediction to the payer, wherein the payer is at least one of a health insurance company, employer, government agency, and government healthcare system.

20. A method of managing medication returns comprising:
providing a medication return platform having a graphical user interface that is remotely accessible by a plurality of users;
receiving, by the medication return platform, medication return data including a piece of patient information and a piece of medication information from a user included in the plurality of users;
verifying, by the medication return platform, an accuracy of the medication return data;
generating, by the medication return platform, a shipping label and a return authorization from for the user;
receiving, from the user, a completed return authorization form and an amount of unused medication sent using the shipping label;
matching, by the medication return platform, the verified medication return data with the amount of unused medication; and
destroying the amount of unused medication.
